(19) United States
(12) Patent Application Publication
(10) Pub. No.: US 2012/0058086 A1
(43) Pub. Date: Mar. 8, 2012

(54) COMPOSITIONS, KITS, AND METHODS FOR PROMOTING ISCHEMIC AND DIABETIC WOUND HEALING

(76) Inventors: Omaida C. Velazquez, Miami, FL (US); Zhao-Jun Liu, Miami, FL (US)

(21) Appl. No.: 13/138,889

(22) PCT Filed: Apr. 9, 2010
(86) PCT No.: PCT/US2010/030471

§ 371 Date: Oct. 18, 2011

Related U.S. Application Data
(60) Provisional application No. 61/171,271, filed on Apr. 21, 2009.

(51) Int. Cl. A61K 38/19; A61P 17/02; A61K 33/00; A61K 35/76; A61K 48/00; A61K 38/17

(52) U.S. Cl. 424/93.2; 514/44 R; 514/18.6; 514/9.4; 424/613

(57) ABSTRACT

Compositions, kits and methods for promoting diabetic wound healing are based on the discovery that SDF-1α specifically upregulates expression of E-selectin in mature endothelial cells (EC), leading to an increase in EC-endothelial progenitor cell (EPC) adhesion and EPC homing. Methods for promoting healing of a wound in a diabetic subject include providing a therapeutically effective amount of a composition including E-selectin protein or a nucleic acid encoding E-selectin protein, and optionally, an agent that specifically upregulates E-selectin expression (e.g., SDF-1α). The methods can also include administering hyperbaric oxygen treatment to the subject. Administering the composition to the subject results in migration of bone marrow-derived progenitor cells to the wound, accelerated wound healing, and upregulation of E-selectin expression in the subject.

### FIELD OF THE INVENTION

The invention relates generally to the fields of medicine and gene therapy. More particularly, the invention relates to compositions, kits and methods for promoting wound healing in diabetic subjects.

### BACKGROUND

Impaired wound healing is a significant clinical problem in diabetic patients and is the leading cause of lower extremity amputation. Current therapies have a limited success rate and fall short in addressing the microvascular pathology present in diabetics. Poor healing of diabetic wounds is characterized by impaired angiogenesis and vasculogenesis. Vasculogenesis involves the growth of neovessels from BM-derived progenitor cells and contributes to the process of postnatal neovascularization and wound healing. The bone marrow (BM)-derived endothelial progenitor cell (EPC) is a key cell involved in vasculogenesis and homes to peripheral tissue in response to ischemia. It remains unknown why the primary physiological stimulus for EPC mobilization and recruitment (i.e., ischemia) fails to induce therapeutic EPC-mediated neovascularization and healing in wounds of diabetic hosts.

Thus, there is currently a need for therapeutic agents and methods for enhancing diabetic would healing.

### SUMMARY

Described herein are compositions, kits and methods for promoting ischemic (e.g., diabetic) wound healing that are based on the discovery that SDF-1α specifically upregulates expression of E-selectin in mature EC, leading to an increase in EC-EPC adhesion and EPC homing. The homing mechanisms of EPC to target tissues involve a cascade of sequential events that include detachment from the bone marrow niche, mobilization into blood vessels and traveling within the circulation, sensing the homing signals, rolling and adhesion onto the endothelial cells (EC) monolayer of the capillary, and subsequent transendothelial migration, in which an EPC-EC direct interaction is required. The experiments described herein investigated whether or not direct cell-cell interactions are required between the EC lining the capillaries and the circulating EPC in order to achieve EPC homing to the target tissues, and if the effect of SDF-1α on EPC homing is mediated, at least partially, by regulating specific adhesion molecule(s) on EC monolayers. An adhesion molecule(s) which mediates SDF-1α-induced homing of EPC, E-selectin, was identified. The results of the experiments described herein demonstrate that SDF-1α specifically upregulates the expression of E-selectin in murine and human mature EC monolayers, that E-selectin is responsible for mediating the effect of SDF-1α on EPC homing by enhancing adhesion of EPC to EC monolayers and their transendothelial migration, and that these effects result in significant enhancement of wound neovascularization and wound healing (neovascularization). These novel findings not only provide an insight into the molecular mechanism (signals) underlying the biological effect of SDF-1α on EPC homing, but also reveal E-selectin as a new target for therapeutic application in ischemic (e.g., diabetic) wound healing.

Unless otherwise defined, all technical terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid), and chemically-modified nucleotides. A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The terms include, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

By the term “gene” is meant a nucleic acid molecule that codes for a particular protein, or in certain cases, a functional or structural RNA molecule.

By the terms “E-selectin gene,” “E-selectin polynucleotide,” “or “E-selectin nucleic acid” is meant a native human E-selectin or E-selectin-encoding nucleic acid sequence, e.g., the native human E-selectin gene (accession no. NM_{—}000450), a nucleic acid having sequences from which an E-selectin cDNA can be transcribed; and/or allelic variants and homologs of the foregoing. The terms encompass double-stranded DNA, single-stranded DNA, and RNA.

By the terms “SDF-1α gene,” “SDF-1α polynucleotide,” “or “SDF-1α nucleic acid” is meant a native human SDF-1α or SDF-1α-encoding nucleic acid sequence, e.g., the native human SDF-1α gene (accession nos. NM_{—}199168, NM_{—}000609, NM_{—}001033886), a nucleic acid having sequences from which an SDF-1α cDNA can be transcribed; and/or allelic variants and homologs of the foregoing. The terms encompass double-stranded DNA, single-stranded DNA, and RNA.

When referring to mutations in a nucleic acid molecule, “silent” changes are those that substitute one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with another amino acid having similar characteristics.

When referring to an amino acid residue in a peptide, oligopeptide or protein, the terms “amino acid residue”, “amino acid” and “residue” are used interchangably and, as used herein, mean an amino acid or amino acid mimetic joined covalently to at least one other amino acid or amino acid mimetic through an amide bond or amide bond mimetic.

As used herein, “protein” and “polypeptide” are used synonymously to mean any peptide-linked chain of amino acids, regardless of length or post-translational modification, e.g., glycosylation or phosphorylation.

By the terms “E-selectin protein” or “E-selectin” is meant an expression product of an E-selectin gene such as the native human E-selectin protein (accession no. AAQ67702, NP_{—}000441.2), or a protein that shares at least 65% (but preferably 75, 80, 85, 90, 95, 96, 97, 98, or 99%) amino acid sequence identity with the foregoing and displays a functional activity of a native E-selectin protein. A “functional activity” of a protein is any activity associated with the physiological function of the protein. For example, functional activities of a native E-selectin protein may include mediating EC-EPC adhesion, and promoting accumulation of blood leukocytes at sites of inflammation by mediating the adhesion of cells to the vascular lining.

By the terms “SDF-1α protein” or “SDF-1α” is meant an expression product of an SDF-1α gene such as the native human SDF-1α protein (accession no. CAG29279.1), or a protein that shares at least 65% (but preferably 75, 80, 85, 90, 95, 96, 97, 98, or 99%) amino acid sequence identity with the foregoing and displays a functional activity of a native SDF-1α protein. A “functional activity” of a protein is any activity associated with the physiological function of the protein. For example, functional activities of a native SDF-1α protein may include, for example, stimulation of cell growth or angiogenesis. SDF-1α is a chemokine that acts as a potent homing signal for EPC (see Lapidot et al., Ann NY Acad Sci 938:83-95, 2001; PCT/US2008/003760).

When referring to a nucleic acid molecule, polypeptide, or infectious pathogen, the term “native” refers to a naturally-occurring (e.g., a wild-type (WT)) nucleic acid, polypeptide, or infectious pathogen.

By “angiogenesis” is meant the growth of new blood vessels originating from existing blood vessels. Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

The terms “specific binding” and “specifically binds” refer to that binding which occurs between such paired species as enzyme/substrate, receptor/agonist, antibody/antigen, etc., and which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

The phrases “isolated” or biologically pure” refer to material which is substantially or essentially free from components which normally accompany it as found in its native state.

The term “antibody” is meant to include polyclonal antibodies, monoclonal antibodies (mAbs), chimeric antibodies, humanized antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques.

As used herein, the terms “chronic wounds” and “problem wounds” and “diabetic wounds” refer to those wounds that do not achieve a sustained anatomic and functional result and that do not heal according to the normal wound healing process of removal of necrotic debris and infection, resolution of inflammation, repair of the connective tissue matrix, angiogenesis, and resurfacing. For example, when hypoxia is pathologically increased, wound healing is impaired and the rate of wound infection increases. An essential part of normal healing is the formation of new vessels within the provisional wound matrix that is referred to as granulation tissue formation.

The term “angiogenesis” refers in one embodiment, to the process by which resident endothelial cells of the wound's adjacent mature vascular network proliferate, and in other embodiments migrate, and remodel into neovessels that grow into the initially avascular wound tissue aided by mature stromal cells such as fibroblasts. In another embodiment, the term “vasculogenesis” refers to a de novo process by which EPC, recruited to the wound, differentiate into endothelial cells and give rise to a replacement vascular network.

By the terms “progenitor cell”, or “endothelial progenitor cells” or “EPC”, is meant any somatic cell which has the capacity to generate fully differentiated, functional progeny by differentiation and proliferation. In another embodiment, progenitor cells include progenitors from any tissue or organ system, including, but not limited to, blood, nerve, muscle, skin, gut, bone, kidney, liver, pancreas, thymus, and the like. Progenitor cells are distinguished from “differentiated cells,” which are defined in another embodiment, as those cells which may or may not have the capacity to proliferate, i.e., self-replicate, but which are unable to undergo further differentiation to a different cell type under normal physiological conditions. In one embodiment, progenitor cells are further distinguished from abnormal cells such as cancer cells, especially leukemia cells, which proliferate (self-replicate) but which generally do not further differentiate, despite appearing to be immature or undifferentiated.

As used herein, the term “totipotent” means an uncommitted progenitor cell such as embryonic stem cell, i.e., both necessary and sufficient for generating all types of mature cells. Progenitor cells which retain a capacity to generate all pancreatic cell lineages but which can not self-renew are termed “pluripotent.” In another embodiment, cells which can produce some but not all endothelial lineages and cannot self-renew are termed “multipotent”.

As used herein, the phrases “bone marrow-derived progenitor cells” and “BM-derived progenitor cells” mean progenitor cells that come from a bone marrow stem cell lineage. Examples of bone marrow-derived progenitor cells include bone marrow-derived mesenchymal stem cells (MSC) and EPC.

The term “homing” refers to the signals that attract and stimulate the cells involved in healing to migrate to sites of injury and aid in repair.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is a non-healing diabetic wound, the result can be healing of the wound (by specifically upregulating E-selectin expression in mature EC leading to increased EC-EPC adhesion, EPC homing and increased wound neovascularization). The compositions and vaccines described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions or vaccines of the invention can include a single treatment or a series of treatments.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, with human patients being preferred. In some cases, the methods of the invention find use in experimental animals, in veterinary applications, and in the development of animal models for disease, including, but not limited to, rodents including mice, rats, and hamsters, as well as non-human primates.

Accordingly, described herein is a method of promoting healing of a diabetic wound in a diabetic subject. The method includes the steps of: providing a therapeutically effective amount of a composition including a pharmaceutically acceptable carrier and at least one therapeutic agent selected from the group consisting of: E-selectin protein, a nucleic acid encoding E-selectin protein, and an agent that specifically upregulates E-selectin expression; and administering the composition to the subject under conditions such that migration of bone marrow-derived progenitor cells (e.g., EPC) to the wound is increased in the subject. The composition can be administered, for example, orally, topically, intravenously, directly to the wound or a site adjacent to the wound, or via endovascular catheter. Administration of the composition to the subject results in accelerated wound healing. In one embodiment, a composition includes E-selectin protein or a nucleic acid encoding E-selectin protein, and an agent that specifically upregulates E-selectin expression, wherein the agent that specifically upregulates E-selectin expression is SDF-1α protein or a nucleic acid encoding SDF-1α protein. The method can further include the step of administering to the subject hyperbaric oxygen treatment.

Further described herein is a method of upregulating E-selectin expression in a diabetic subject having a diabetic wound. The method includes administering to the diabetic subject a composition including at least one rAAV virion comprising a polynucleotide encoding E-selectin, the polynucleotide interposed between a first AAV inverted terminal repeat and second AAV inverted terminal repeat, the composition in an amount effective to upregulate E-selectin expression, induce migration of bone marrow-derived progenitor cells (e.g., EPC) to the wound, and accelerate healing of the wound in the subject. The at least one rAAV virion can include serotype 2 capsid proteins. The composition can be administered, for example, directly to the wound or a site adjacent to the wound. The method can further include the step of administering SDF-1α protein or a nucleic acid encoding SDF-1α protein to the subject and/or administering to the subject hyperbaric oxygen treatment.

Yet further described herein is a kit for treating at least one diabetic wound in a mammalian subject. The kit includes a therapeutically effective amount of a composition including a pharmaceutically acceptable carrier and at least one therapeutic agent selected from the group consisting of: E-selectin protein, a nucleic acid encoding E-selectin protein, and an agent that specifically upregulates E-selectin expression; and instructions for use. In one embodiment, the composition includes E-selectin protein or a nucleic acid encoding E-selectin protein, and an agent that specifically upregulates E-selectin expression, wherein the agent that specifically upregulates E-selectin expression is SDF-1α protein or a nucleic acid encoding SDF-1α protein. In one embodiment, the instructions for use include instructions for administering hyperbaric oxygen treatment to the subject.

Also described herein is a method of promoting healing of a diabetic wound in a diabetic subject. The method includes the steps of: providing a composition including a pharmaceutically acceptable carrier and a plurality of bone marrow-derived progenitor cells, wherein the bone marrow-derived progenitor cells comprise a polynucleotide encoding E-selectin; and administering the composition to the subject in an amount effective to increase migration of bone marrow-derived progenitor cells (e.g., EPC) to the wound and accelerate healing of the wound in the subject. The polynucleotide encoding E-selectin can be within a viral vector, e.g., a viral vector included within a viral particle (e.g., an rAAV vector within an AAV particle). A composition can further include SDF-1α protein or a nucleic acid encoding SDF-1α protein, and can be administered directly to the wound or a site adjacent to the wound. In one embodiment, the method further includes the step of administering to the subject hyperbaric oxygen treatment.

Although compositions, kits and methods similar or equivalent to those described herein can be used in the practice or testing of the present invention, suitable compositions, kits and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a series micrographs of cells and graphs showing that SDF-1α engineered BMDFs promote neovascularization and diabetic wound healing. (A) Left: wound healing rate expressed as percent recovery. Two groups of NOD mice were wounded and treated with mSDF-1α/BMDFs versus GFP/BMDFs. The fraction of initial wound area was measured daily by digital photography and ImageJ analysis until wounds were healed. Diabetic mice treated with mSDF-1α/BMDFs had significantly improved wound closure rates from day 3 when compared with GFP/BMDFs treated controls. Right: representative wounds at different days are shown for each group. (B) Wound blood vessel perfusion with Dil dye. Upper: representative images of Dil-stained wound blood vessels measured by laser scanning confocal microscopy at day 5 are shown for each group. Lower: Quantification of vessel density in the wounds. Percentage of threshold area covers all vessels detected as a percent of the entire wound area. mSDF-1α/BMDFs treated wounds had significantly higher vessel density compared to the control. Data are presented as mean±SD of four wounds in each group. (C) IHC of wound tissues injected with mSDF-1α/BMDFs versus GFP/BMDFs for the detection of mSDF-1α. Stronger mSDF-1α expression (brown) was observed in mSDF-1α/BMDFs-treated wounds compared to the GFP/BMDFs treated wounds (40X).

FIG. 2 is a photograph of EC monolayers and a graph showing that increase adhesion of EPC to SDF-1α stimulated EC monolayer in vitro. Dil-Ac-LDL-labeled EPC were added to EC monolayers which were stimulated with SDF-1α or BSA. After 30 min, unbound EPC were washed out. Bound EPC were quantified by fluorescence scanning. (A): representative images. (B): quantitative data. Data are presented as mean±SD of three independent assays in which samples were duplicated.

FIG. 3 is a graph, a photograph of an electrophoretic gel, a series of micrographs of NOD mice wounds, and a micrograph of wound tissue showing that SDF-1α stimulation up-regulates expression of E-selectin in EC monolayers. (A) HMVEC were stimulated with SDF-1α or BSA for 4 hours and total RNA were extracted. Expression of extracellular matrix and adhesion molecules were analyzed using RT2 PCR Array. Expression of E-selectin was upregulated by SDF-1α stimulation. Levels of mRNA in BSA-treated EC were set as “1” and compared to that in SDF-1α treated EC. (B): Expression of E-selectin was validated by Western blot analysis. HMVECs were stimulated with SDF-1α or BSA and cells were harvested at various time points. β-actin was used as loading control. (C) Increased vascular expression of E-selectin in NOD mice wounds injected with mSDF-1α/AAV compared to that injected with LacZ/AAV. Co-expression (yellow) of KDR (red) and E-selectin (green) in vessels was detected by immunostaining. (D) Increased expression of SDF-1α in wound tissues-injected with mSDF-1α/AAV was detected by IHC.

FIG. 4 is a pair of graphs and a series of micrographs of EC showing that SDF-1α induced E-selectin in an EC monolayer increases EPC adhesion and transendothelial migration. (A) More EPC adhered to SDF-1α-stimulated EC monolayer than BSA-treated EC monolayer. Addition of blocking Ab against E-selectin inhibited the interaction of SDF-1α-stimulated EC with EPC in this cell-cell adhesion assay. Data are presented as mean±SD of three independent assays in which samples were duplicated. (B, upper panel) Increased numbers of Dil-Ac-LDL labeled EPC were observed to transmigrate to the lower chamber of the transwell with SDF-1α-stimulated EC monolayer than with BSA-treated EC monolayer. Blocking Ab against E-selectin inhibited this EPC transendothelial migration. (B, lower panel) Dil-EPCs on the lower chamber of the transwell were quantified by fluorescence scanner. Blocking Ab against E-selectin inhibited interaction of SDF-1α-stimulated EC with EPC. Data are presented as mean±SD of three independent assays in which samples were duplicated.

FIG. 5 is a photograph of a Western blot demonstrating that SDF-1α induces expression of E-selectin ligands in EPC. EPC were stimulated by SDF-1α and cells were harvested at various time points. Expression of E-selectin ligands, CD162 and CD44, was examined by Western blotting assay. β-actin was used as loading control. Experiments were repeated twice.

FIG. 6 is a series of photographs and graphs showing the involvement of E-selectin in SDF-1α-induced EPC homing, neovascularization and wound healing in murine model of hindlimb ischemia plus cutaneous wounding. (A) Upper: representative images of non-invasive LDI measurements showing spontaneous restoration of blood flow into ischemic hindlimbs after femoral artery ligation/excision in E-sel^{-/-} versus WT mice. Lower: Quantitative data of LDI measurements. Ratio of ischemic versus normal hindlimb between two groups of mice at various time points. Data are presented as mean±SD from each group (n=6/group). (B) Wound closure rates in E-sel^{-/-} versus WT. Upper: representative images of wound healing in E-sel^{-/-} and WT mice. Deletion of E-selectin delayed wound healing. Lower: Quantitative wound closure rates in E-sel^{-/-} versus WT mice. Data are presented as percentage wound closure (recovery), mean±SD from each group (n=6/group). (C) Wound blood vessel perfusion with Dil dye. Upper: representative images of Dil-stained wound blood vessels detected by confocal laser scanning photography at day 7 are shown for each group. Lower: Software-assisted quantification of vessel density in the entire area of residual wounds at day 7, as percent fluorescence. Wounds from WT mice had significantly higher vessel density compared to that from E-sel^{-/-} mice. Data are presented as mean±SD of three wounds in each group. (D) E-sel is essential for EPC homing to the wound lesion. 1×10⁷ of bone marrow cells from ROSA26 (LacZ⁺) mice were transplanted to E-sel^{-/-} and WT mice, respectively. Ischemic hindlimb wounds were created and SDF-1α was injected into wounds. 7 days after cell transplantation, wounds were harvested and frozen samples were subjected for X-gal (blue) and anti-KDR (brown) staining. Upper: representative images of double staining. Lower: number of double positive cells was counted from 5 randomly selected fields in wound samples. Data are percentage of mean±SD from each group (n=3 wounds/group).

### DETAILED DESCRIPTION

The compositions, methods and kits described herein are based on the discovery of signals that modulate mature SDF-1α-mediated EPC homing by EC and circulating EPC. In the experiments described below, increasing levels of SDF-1α in a wound via cell-based therapy was shown to promote healing in diabetic mice (−20% increase healing rates by day 3, P=0.006). SDF-1α increased EC-EPC adhesion and specifically, upregulated E-selectin expression in human microvascular ECs (4.6-fold increase, P<0.01). This effect was also significant in blood vessels of the experimental mice and resulted in increased wound neovascularization. The regulatory effects of SDF-1α on EC-EPC adhesion and EPC homing were specifically mediated by E-selectin, as the application of E-selectin antagonists significantly inhibited SDF-1α-induced EC-EPC adhesion, EPC homing, wound neovascularization, and wound healing. Requirement of E-selectin in mediating SDF-1α-induced wound healing is demonstrated in E-sel^{-/-} mouse model. SDF-1α-engineered cell-based therapy promotes diabetic wound healing in mice by specifically, upregulating E-selectin expression in mature ECs leading to increase EC-EPC adhesion and EPC homing and increased wound neovascularization. These findings provide novel insight into the signals underlying the biological effect of SDF-1α on EPC homing and point to E-selectin as a new potential target for therapeutic manipulation of EPC traffic in ischemic (e.g., diabetic) wound healing.

The below described preferred embodiments illustrate adaptations of these compositions, vaccines, kits and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fl, 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988. Conventional methods of gene transfer and gene therapy may also be adapted for use in the present invention. See, e.g., Gene Therapy: Principles and Applications, ed. T. Blackenstein, Springer Verlag, 1999; and Gene Therapy Protocols (Methods in Molecular Medicine), ed. P. D. Robbins, Humana Press, 1997.

### Compositions For Promoting Ischemic Wound Healing

Compositions for promoting healing of an ischemic (e.g., diabetic) wound in a diabetic subject are described herein. The compositions described herein can be used for promoting healing of any type of ischemic wound, such as a diabetic wound. Such compositions generally include a therapeutically effective amount of a composition including a pharmaceutically acceptable carrier and at least one therapeutic agent such as E-selectin protein or a nucleic acid encoding E-selectin protein. A composition can further include an agent that specifically upregulates E-selectin expression. In this embodiment, any suitable agent that specifically upregulates E-selectin expression can be used. For example, SDF-1α can be used. Additional examples include IL-1, TNF-alpha and lipopolysaccharide (LPS). In this embodiment, a composition includes E-selectin protein or a nucleic acid encoding E-selectin protein, and SDF-1α protein or a nucleic acid encoding SDF-1α protein. Administration of the composition to the subject results in increased migration of bone marrow-derived progenitor cells to the wound and accelerated wound healing in the subject. Wound healing is characterized by re-epithelization of the wound.

E-selectin protein can be isolated or synthesized using any suitable protocol (e.g., Protein A and Protein G beads-based anti-E-selectin antibody capture technique (Invitrogen)). In a typical embodiment of administering E-selectin protein, the E-selectin protein is prepared/synthesized by transforming bacterial cells (e.g., E. Coli) or transfecting mammalian cells, and then purifying E-selectin from the cells. In an embodiment in which SDF-1α protein is also administered, SDF-1α protein is similarly prepared.

In another embodiment, a nucleic acid encoding E-selectin can be administered to a subject for treating a diabetic wound(s). The coding sequence which encodes E-selectin may be identical to the nucleotide sequence of accession no. NM_{—}000450, or it may also be a different coding sequence which, as a result of the redundancy or degeneracy of the genetic code, encodes the same polypeptide as the polynucleotide of accession no. NM_{—}000450. Other nucleic acid molecules as described herein include variants of the native E-selectin gene such as those that encode fragments, analogs and derivatives of a native E-selectin protein. Such variants may be, e.g., a naturally occurring allelic variant of the native E-selectin gene, a homolog of the native E-selectin gene, or a non-naturally occurring variant of the native E-selectin gene. These variants have a nucleotide sequence that differs from the native E-selectin gene in one or more bases. For example, the nucleotide sequence of such variants can feature a deletion, addition, or substitution of one or more nucleotides of the native E-selectin gene.

In other embodiments, variant E-selectin proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histadine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of a native E-selectin gene or native E-selectin mRNAs as described herein are nucleic acids isolated from human tissue that have at least 75% (e.g., 76%, 77%, 78%, 79%, 80%, 81%, 82%, 83%, 84%, 85%, 86%, 87%, 88%, 89%, 90%, 92%, 93%, 94%, 95%, 96%, 97%, 98%, and 99%) sequence identity with the native E-selectin gene or native E-selectin mRNAs, and encode polypeptides having structural similarity to a native E-selectin protein. Homologs of the native E-selectin gene or native E-selectin mRNAs as described herein are nucleic acids isolated from other species that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human E-selectin gene or native human E-selectin mRNAs, and encode polypeptides having structural similarity to native human E-selectin protein. Public and/or proprietary nucleic acid databases can be searched to identify other nucleic acid molecules having a high percent (e.g., 70, 80, 90% or more) sequence identity to the native E-selectin gene or native E-selectin mRNAs.

Non-naturally occurring E-selectin gene or mRNA variants are nucleic acids that do not occur in nature (e.g., are made by the hand of man), have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human E-selectin gene or native human E-selectin mRNAs, and encode polypeptides having structural similarity to native human E-selectin protein. Examples of non-naturally occurring E-selectin gene variants are those that encode a fragment of a E-selectin protein, those that hybridize to the native E-selectin gene or a complement of the native E-selectin gene under stringent conditions, those that share at least 65% sequence identity with the native E-selectin gene or a complement thereof, and those that encode a E-selectin fusion protein.

Nucleic acids encoding fragments of a native E-selectin protein as described herein are those that encode, e.g., 2, 5, 10, 25, 50, 100, 150, 200 or more amino acid residues of the native E-selectin protein. Shorter oligonucleotides (e.g., those of 6, 7, 8, 9, 10, 11, 12, 13, 14, 15, 16, 17, 18, 19, 20, 30, 50, base pairs in length) that encode or hybridize with nucleic acids that encode fragments of a native E-selectin protein can be used as probes, primers, or antisense molecules. Nucleic acids encoding fragments of a native E-selectin protein can be made by enzymatic digestion (e.g., using a restriction enzyme) or chemical degradation of the full length native E-selectin gene, a E-selectin mRNA or cDNA, or variants of the foregoing. Using the nucleotide sequence of the native human E-selectin gene and the amino acid sequence of the native E-selectin protein previously reported, those skilled in the art can create nucleic acid molecules that have minor variations in their nucleotide sequence, by, for example, standard nucleic acid mutagenesis techniques or by chemical synthesis. Variant E-selectin nucleic acid molecules can be expressed to produce variant E-selectin proteins.

### Methods of Promoting Diabetic Wound Healing

One embodiment of a method of promoting healing of a diabetic wound in a diabetic subject includes providing a therapeutically effective amount of a composition including a pharmaceutically acceptable carrier and at least one therapeutic agent such as E-selectin protein, a nucleic acid encoding E-selectin protein, and an agent that specifically upregulates E-selectin expression; and administering the composition to the subject under conditions such that migration of bone marrow-derived progenitor cells (e.g., EPC) to the wound is increased in the subject. For example, a composition can include E-selectin protein or a nucleic acid encoding E-selectin protein, and an agent that specifically upregulates E-selectin expression, such as SDF-1α protein or a nucleic acid encoding SDF-1α protein. In this method, the composition can be administered by any suitable route, e.g., orally, topically, intravenously, or directly to the wound or a site adjacent to the wound. Administration of the composition to the subject results in accelerated wound healing.

A method of healing a diabetic wound in a diabetic subject can further include administering to the subject hyperbaric oxygen treatment. In the methods described herein, hyperbaric oxygen therapy (HBO₂) is typically an adjunctive therapy used to stimulate wound healing in situations where the microvasculature has become attenuated. Methods of treating a diabetic subject with HBO₂ are described, for example, in PCT/US2008/003760 which is incorporated herein by reference. In one example of a method of promoting wound healing in a diabetic subject (patient), patients receive 20 or more treatments breathing 100% O₂ in a pressurized chamber at between about 2.0 to about 3.2 atmospheres absolute (ATA), once or twice daily. Treatment time ranges are generally from about 10 minutes to about 240 minutes (e.g., about 10, 15, 30, 60, 90, 120, 150, 180, 210, 240, etc. minutes).

In another embodiment, a method of upregulating E-selectin expression in a diabetic subject having a diabetic wound includes administering to the diabetic subject a composition including at least one rAAV virion including a polynucleotide encoding E-selectin, the polynucleotide interposed between a first AAV inverted terminal repeat and second AAV inverted terminal repeat. In this embodiment, the composition is in an amount effective to upregulate E-selectin expression, induce migration of bone marrow-derived progenitor cells (e.g., EPC) to the wound, and accelerate healing of the wound in the subject. In this method, the at least one rAAV virion can include serotype 2 capsid proteins. As in the other embodiments described herein, the composition can be administered by any suitable route, e.g., orally, topically, intravenously, or directly to the wound or a site adjacent to the wound. This method can further include administering SDF-1α protein or a nucleic acid encoding SDF-1α protein to the subject, and/or the step of administering hyperbaric oxygen treatment to the subject.

In yet another embodiment, a method of promoting healing of a diabetic wound in a diabetic subject includes the steps of: providing a composition including a pharmaceutically acceptable carrier and a plurality of bone marrow-derived progenitor cells, and administering the composition to the subject in an amount effective to increase migration of bone marrow-derived progenitor cells (e.g., EPC) to the wound and accelerate healing of the wound in the subject. In this embodiment, the bone marrow-derived progenitor cells include a polynucleotide encoding E-selectin. The polynucleotide encoding E-selectin can be included within a viral vector, such as an rAAV vector. Typically, the rAAV vector is within an rAAV virus (particle). In this embodiment, the composition can further include SDF-1α protein or a nucleic acid encoding SDF-1α protein. If the composition includes a nucleic acid encoding SDF-1α, the nucleic acid can be present within the rAAV vector, within a second rAAV vector, or within a viral vector other than rAAV. The composition(s) can be administered by any suitable route, e.g., orally, topically, intravenously, or directly to the wound or a site adjacent to the wound. The method can further include the step of administering to the subject hyperbaric oxygen treatment.

The methods described herein can be used to treat a number of different types of wounds. One example of a diabetic wound is a livedoid vasculopathy, a disorder characterised by painful ulceration in association with livedo reticularis and atrophie blanche. Another example of a diabetic wound is a diabetic ulcer, e.g., a peripheral arterial disease ulcer, a venous stasis ulcer, a chronic non-healing ulcer, a pressure ulcer, decubitus ulcers, chronic foot ulcers, etc. A wound can also be a combination of one or more of the above-listed wounds. A diabetic subject may have one or multiple wounds to be treated. Subjects include any mammal such as human beings, rats, mice, cats, dogs, goats, sheep, horses, monkeys, apes, rabbits, cattle, etc. The subject (e.g., mammal) can be in any stage of development including adults, and juveniles. Target tissues can be any within the subject such as retina, liver, kidney, heart, lungs, components of gastrointestinal tract, pancreas, gall bladder, urinary bladder, the central nervous system including the brain, skin, bones, etc.

In methods in which a nucleic acid encoding E-selectin (and optionally a nucleic acid encoding SDF-1α or other agent for modulating EPC homing) is administered to a diabetic subject, a nucleic acid as described herein can also include one or more expression control sequences operatively linked to the nucleic acid encoding E-selectin (and optionally a nucleic acid encoding SDF-1α or other agent for modulating EPC homing). Numerous such sequences are known. Those to be included can be selected based on their known function in other applications. Examples of expression control sequences include promoters, insulators, silencers, response elements, introns, enhancers, initiation sites, termination signals, and pA tails.

To achieve appropriate levels of E-selectin (and optionally SDF-1α), any of a number of promoters suitable for use in the target cells may be employed. For example, constitutive promoters of different strengths can be used. Expression vectors and plasmids as described herein may include one or more constitutive promoters, such as viral promoters or promoters from mammalian genes that are generally active in promoting transcription. Examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Examples of constitutive mammalian promoters include various housekeeping gene promoters, as exemplified by the β-actin promoter.

Inducible promoters and/or regulatory elements may also be contemplated for use in the compositions and methods described herein. Examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are useful in certain embodiments of the compositions and methods described herein. Examples of such promoters that may be used with expression vectors expression vectors as described herein include Tie-2 or KDR_promoter.

Compositions as described herein (e.g., compositions including a viral vector encoding E-selectin) may be administered to a mammalian subject by any suitable technique. Various techniques using viral vectors for the introduction of an E-selectin gene into cells are provided for according to the compositions and methods described herein. Viruses are naturally evolved vehicles which efficiently deliver their genes into host cells and therefore are desirable vector systems for the delivery of therapeutic genes. Preferred viral vectors exhibit low toxicity to the host cell and produce therapeutic quantities of E-selectin protein (e.g., in a tissue-specific manner). Viral vector methods and protocols are reviewed in Kay et al. Nature Medicine 7:33-40, 2001.

Although the experiments described below involve rAAV and lentivirus, any suitable viral vector can be used. Many viral vectors are known in the art for delivery of genes to mammalian subject and a non-exhaustive list of examples follows. Methods for use of recombinant Adenoviruses as gene therapy vectors are discussed, for example, in W. C. Russell, Journal of General Virology 81:2573-2604, 2000, and Bramson et al., Curr. Opin. Biotechnol. 6:590-595, 1995. Methods for use of Herpes Simplex Virus vectors are discussed, for example, in Cotter and Robertson, Curr. Opin. Mol. Ther. 1:633-644, 1999. Replication-defective lentiviral vectors, including HIV, may also be used. Methods for use of lentiviral vectors are discussed, for example, in Vigna and Naldini, J. Gene Med. 5:308-316, 2000 and Miyoshi et al., J. Virol. 72:8150-8157, 1998. Retroviral vectors, including Murine Leukemia Virus-based vectors, may also be used. Methods for use of retrovirus-based vectors are discussed, for example, in Hu and Pathak, Pharmacol. Rev. 52:493-511, 2000 and Fong et al., Crit. Rev. Ther. Drug Carrier Syst. 17:1-60, 2000. Other viral vectors that may find use include Alphaviruses, including Semliki Forest Virus and Sindbis Virus. Hybrid viral vectors may be used to deliver an E-selectin gene to a target tissue (e.g., a diabetic wound). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology.

In some embodiments, nucleic acids of the compositions and methods described herein are incorporated into rAAV vectors and/or virions in order to facilitate their introduction into a cell. Useful rAAV vectors are recombinant nucleic acid constructs that include (1) a heterologous sequence to be expressed (e.g., a polynucleotide encoding an E-selectin protein) and (2) viral sequences that facilitate integration and expression of the heterologous genes. The viral sequences may include those sequences of AAV that are required in cis for replication and packaging (e.g., functional ITRs) of the DNA into a virion. In typical applications, the heterologous gene encodes E-selectin, which is useful for increasing bone marrow-derived progenitor cell migration to a wound and promoting wound healing in a diabetic subject. Such rAAV vectors may also contain marker or reporter genes. Useful rAAV vectors have one or more of the AAV WT genes deleted in whole or in part, but retain functional flanking ITR sequences. The AAV ITRs may be of any serotype (e.g., derived from serotype 2) suitable for a particular application. Methods for using rAAV vectors are discussed, for example, in Tal, J., J. Biomed. Sci. 7:279-291, 2000 and Monahan and Samulski, Gene delivery 7:24-30, 2000.

The nucleic acids and vectors of the invention can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described. See, e.g., U.S. Pat. Nos. 5,173,414, 5,139,941, 5,863,541, and 5,869,305, 6,057,152, 6,376,237; Rabinowitz et al., J. Virol. 76:791-801, 2002; and Bowles et al., J. Virol. 77:423-432, 2003.

rAAV virions useful in the invention include those derived from a number of AAV serotypes including 1, 2, 3, 4, 5, 6, 7, 8 and 9. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

Also useful in the compositions, kits and methods described herein are pseudotyped rAAV. Pseudotyped vectors include AAV vectors of a given serotype pseudotyped with a capsid gene derived from a serotype other than the given serotype. Techniques involving the construction and use of pseudotyped rAAV virions are known in the art and are described in Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet. 10:3075-3081, 2001.

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45, 2000. Other rAAV virions that can be used in methods and compositions as described herein include those capsid hybrids that are generated by molecular breeding of viruses as well as by exon shuffling. See Soong et al., Nat. Genet. 25:436-439, 2000; and Kolman and Stemmer Nat. Biotechnol. 19:423-428, 2001.

Parenteral administration of viral vectors or virus particles by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the vectors or virus particles may be in powder form (e.g., lyophilized) for constitution with a suitable vehicle, for example, sterile pyrogen-free water, before use.

In addition to viral vectors, any suitable vehicle or vector for introducing a nucleic acid encoding E-selectin into a diabetic subject having at least one diabetic wound can be used. For example, ultrasound-based gene delivery technology can be used. Additional examples include particle bombardment and cell electropermeabilization. Synthetic gene transfer molecules that form multicellular aggregates with plasmid DNA are also useful. Such molecules include polymeric DNA-binding cations (Guy et al., Mol. Biotechnol. 3:237-248, 1995), cationic amphiphiles (lipopolyamines and cationic lipids, Felgner et al., Ann. NY Acad. Sci. 772:126-139, 1995), and cationic liposomes (Fominaya et al., J. Gene Med. 2:455-464, 2000).

In some embodiments, EPC including rAAV-E-selectin vectors or viruses are administered to a diabetic subject in order to treat one or more diabetic wounds. Several approaches may be used for the introduction of EPC into the subject, including catheter-mediated delivery I.V. (e.g., endovascular catheter), or direct injection into a target site, e.g, a diabetic wound. Techniques for the isolation of donor stem cells and transplantation of such isolated cells are known in the art. Ex vivo delivery of cells transduced with rAAV virions is also encompassed by the methods described herein. Ex vivo gene delivery may be used to transplant, for example, rAAV-transduced host cells (e.g., EPC) back into the host. A suitable ex vivo protocol may include several steps. A segment of target tissue (e.g., BM-derived EPCs) may be harvested from the host and rAAV virions may be used to transduce an E-selecting-encoding nucleic acid into the subject's (i.e., host's) cells. These genetically modified cells may then be transplanted back into the subject. Several approaches may be used for the reintroduction of cells into the subject, including intravenous injection, intraperitoneal injection, or in situ injection into target tissue. Microencapsulation of cells transduced or infected with rAAV modified ex vivo is another technique that may be used. Autologous as well as allogeneic cell transplantation may be used according to the invention.

In order to increase recruitment of BM-derived progenitor cells from the BM to a non-BM compartment (e.g., the target tissue), compositions for promoting wound healing as described herein can include, in addition to E-selection and SDF-1α, any other agent capable of promoting recruitment of BM-derived progenitor cells. A number of such agents are known. See, e.g., those described in International Application WO 00/50048; integrins (e.g., α4, α5), selectin family of adhesion molecules, VCAM-1, and colony stimulating factors.

The therapeutic methods described herein in general include administration of a therapeutically effective amount of the compositions described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider. The methods and compositions herein may be also used in the treatment of any other disorders in which downregulation of E-selectin signaling, expression, or activity may be implicated.

In one embodiment, a method of promoting healing of a diabetic wound in a diabetic subject includes monitoring treatment progress. Monitoring treatment progress in a subject generally includes determining a measurement of wound size or other diagnostic measurement in a subject having a diabetic wound(s), prior to administration of a therapeutic amount of a composition as described herein sufficient to promote healing of the wound to the subject. At one or more time points subsequent to the subject having been administered a therapeutic amount of a composition as described herein sufficient to promote healing, a second measurement of wound size is determined and compared to the first measurement of wound size. The first and subsequent measurements are compared to monitor the course of wound healing (e.g., decrease in wound size) and the efficacy of the therapy.

### Kits

Described herein are kits for treating at least one diabetic wound in a mammalian subject. A typical kit includes a therapeutically effective amount of a composition including a pharmaceutically acceptable carrier and at least one therapeutic agent such as E-selectin protein, a nucleic acid encoding E-selectin protein, or an agent that specifically upregulates E-selectin expression, and instructions for use. In one embodiment, a kit includes a therapeutic composition containing a therapeutically effective amount of an E-selectin protein or a nucleic acid encoding E-selectin protein, as well as an agent that specifically upregulates E-selectin expression and instructions for use. For example, a kit for treating at least one diabetic wound in a mammalian subject includes a therapeutic composition containing a therapeutically effective amount of E-selectin protein or a nucleic acid encoding E-selectin (e.g., rAAV-E-selectin) and a therapeutically effective amount of SDF-1α for promoting wound healing and a pharmaceutically acceptable carrier in unit dosage form. Generally, a kit as described herein includes packaging and instructions for use. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In an embodiment in which the subject to be treated is administered hyperbaric oxygen treatment in addition to a therapeutic composition as described herein, the instructions for use will typically include instructions for administering hyperbaric oxygen treatment to the subject.

In an embodiment in which EPC (e.g., EPC including rAAV-E-selectin) are to be administered to a diabetic subject, a kit as described herein includes a therapeutic amount of EPC together with instructions for administering the cells to the subject having one or more diabetic wounds. The cells can be packaged by any suitable means for transporting and storing cells; such methods are well known in the art. The instructions generally include one or more of: a description of the cells; dosage schedule and administration for treatment of a diabetic wound; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Administration of Compositions

The compositions of the invention may be administered to mammals (e.g., rodents, humans) in any suitable formulation. For example, a composition for promoting wound healing as described herein (e.g., E-selectin, a nucleic acid encoding E-selectin, SDF-1α) may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to mammals by any conventional technique. Typically, such administration will be topical (e.g., aerosol, cream, foam, gel, liquid, ointment, paste, powder, shampoo, spray, patch, disk, or dressing) or oral. When compositions are formulated in one embodiment for oral delivery, the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Alternatively, administration may be parenteral (e.g., intravenous, subcutaneous, intratumoral, intramuscular, intraperitoneal, or intrathecal introduction). Compositions may also be provided herein via an intradermal patch, i.e., the patch is administered adjacent to the area of skin to be treated. As used herein a “patch” includes at least the compositions provided herein and a covering layer, such that, the patch can be placed over the area of skin to be treated. The patch can be designed to maximize delivery of the compositions provided herein through the stratum corneum and into the epidermis or dermis, reduce lag time, promote uniform absorption, and reduce mechanical rub-off.

The compositions may also be administered directly to a target site by, for example, surgical delivery to an internal or external target site, or by catheter (e.g., endovascular catheter) to a site accessible by a blood vessel. When treating a subject having a diabetic wound, the composition may be administered to the subject intravenously, directly into the wound, or to the wound surface. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

Pharmaceutical compositions as described herein may be formulated to release the therapeutic agent (e.g., E-selectin protein, nucleic acid encoding E-selectin, agent that specifically upregulates E-selectin expression) substantially immediately upon administration or at any predetermined time or time period after administration. The latter types of compositions are generally known as controlled release formulations. Formulations that create a substantially constant concentration of the drug within the body over an extended period of time can be used. In another embodiment, formulations that after a predetermined lag time create a substantially constant concentration of the therapeutic agent within the body over an extended period of time can be used. As another example, formulations that sustain action during a predetermined time period by maintaining a relatively, constant, effective level in the body with concomitant minimization of undesirable side effects associated with fluctuations in the plasma level of the active substance can be used. Also finding use in the compositions described herein are formulations that allow for convenient dosing, such that doses are administered, for example, once every one or two weeks, as well as formulations that target a diabetic wound by using, for example, osmotic pumps or ultrasound-based gene delivery technology to deliver the therapeutic agent to a particular cell type (e.g., a endothelial cells).

A number of strategies are available for obtaining controlled release in which the rate of release outweighs the rate of metabolism of the therapeutic agent being delivered. In one example, controlled release is obtained by appropriate selection of various formulation parameters and ingredients, including, e.g., various types of controlled release compositions and coatings. The therapeutic can be formulated with appropriate excipients into a pharmaceutical composition that, upon administration, releases the therapeutic agent in a controlled manner. Examples include single or multiple unit tablet or capsule compositions, oil solutions, suspensions, emulsions, microcapsules, microspheres, molecular complexes, nanoparticles, patches, and liposomes.

### Effective Doses

The compositions described above are preferably administered to a mammal (e.g., human) in an effective amount, that is, an amount capable of producing a desirable result in a treated mammal (e.g., promoting diabetic wound healing in mice by specifically upregulating E-selectin expression in mature EC leading to increase EC-EPC adhesion, EPC homing and increased wound neovascularization.). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently. It is expected that an appropriate dosage for intravenous administration of particles would be in the range of, for example, about 10⁹ virus particles per wound, and an appropriate dosage for recombinant protein (e.g., SDF-1α, E-selectin), for example, is 25 μg/kg.

### EXAMPLES

The present invention is further illustrated by the following specific examples. The examples are provided for illustration only and should not be construed as limiting the scope of the invention in any way.

### Example 1

### E-selectin Mediates Endothelial Progenitor Cell Homing in Response to SDF-1α and Mediates Normal Limb Revascularization and Wound Healing Rates: Potential Clinical Utility for Non-Healing Diabetes-Associated Microangiopathy and Non-Healing Wounds

SDF-1α serves as a homing signal for recruitment of the EPC from bone marrow to peripheral areas in need of neovascularization (Gallagher et al., SDF-1α as a critically important factor, deficient in Diabetes-associated non-healing cutaneous wounds: J Clin Invest 2007, 117:1249-1259), but the mechanism whereby SDF-1α results in homing of the circulating EPC to the target tissues has not heretofore been discovered. It was hypothesized that direct cell-cell interactions are required between the mature EC lining the capillary and the circulating EPC in order to achieve EPC homing to the target tissues and that such interactions impact the rate of limb revascularization and cutaneous wound healing. Described herein is the identification of the specific adhesion molecule regulated by SDF-1α on the mature EC that mediate the EPC homing process; this novel data from studies in mice and human cells identified this molecule as E-Selectin. Additional data indicated that limb revascularization and wound healing are favorably impacted by tissue-level E-Selectin expression. It was also hypothesized that E-Selectin locally delivered to non-healing wounds that suffer from microangiopathy (such as the non-healing wounds in patients with Diabetes) will carry utility as a new wound-healing technology. An AAV vector encoding E-Selectin that may be tested in ischemic and diabetic wounds is under development.

Using RT² Profiler PCR Arrays, the extracellular matrix and adhesion molecules induced by SDF-1α were studied in cultured human mature ECs. Molecule(s) found to have significantly increased gene expression by the arrays were validated by Western blotting and immunohistochemistry. The effects of SDF-1α on direct cell-cell interactions between human EPC to EC were investigated by cell adhesion and trans-endothelial migration assays. The specific role of identified adhesion molecule(s) in mediating the SDF-1α-induced EC-EPC interaction, EPC homing, limb revascularization, and wound healing was studied using antagonists in vitro and in vivo. Bone marrow transplantation experiments (bone marrow cells from Rasa26 mice were transplanted into E-sel^{+/+} vs E-sel^{-/-} mice) were carried out to study the recruitment of EPC to wounds and the wound healing rates, with and without tissue level expression of E-Selectin. Data was analyzed by ANOVA.

SDF-1α significantly increased EC-EPC adhesion and trans-endothelial migration in vitro and enhanced EPC homing in vivo. SDF-1α specifically upregulated expression of E-Selectin, but not P- and L-selectin in cultured human mature microvascular ECs and in blood vessels of experimental mice. The regulatory effects of SDF-1α on EC-EPC adhesion and EPC homing were specifically mediated by E-selectin, as the application of E-selectin antagonists significantly inhibited SDF-1α-induced EC-EPC adhesion, transmigration and EPC homing. Tissue level expression of E-selectin is required for normal wound healing and neovascularization, in vivo and impacts favorably on these two biologic events that are critical for normal healing and defective in Diabetes-associated non-healing wounds.

In conclusion, SDF-1α specifically upregulated expression of E-selectin in mature ECs leading to increase EC-EPC adhesion, EPC homing, limb revascularization, and wound healing. These novel findings provide profound insight into the molecular mechanism underlying the biological effect of SDF-1α on EPC homing and diabetic wound healing and by direct corollary point to E-selectin as a new target for therapeutic manipulation of EPC traffic, neovascularization, and wound healing in Diabetes-associated microangiopathy and non-healing cutaneous wounds.

### Example 2

### Identification of E-Selectin as a Novel Target for the Regulation of Post-Natal Neovascularization: Implications for Diabetic Wound Healing

It was previously reported that SDF-1α, a homing signal for recruiting EPC to areas of neovascularization, is down-regulated in diabetic wounds (Gallagher et al., J Clin Invest 2007, 117:1249-1259). In the experiments described below, signals whereby mature EC and circulating EPC achieve SDF-1α-mediated EPC homing were investigated. SDF-1α in diabetic wounds were therapeutically increased by injection of SDF-1α engineered bone marrow-derived fibroblasts versus control cells (N=48 (20, NOD), (28, STZ-057)). PCR-array gene expression differences were validated by Western blotting and immunohistochemistry. The role of adhesion molecule(s) in mediating SDF-1α-induced EPC homing and wound healing was furthered studied using antagonists in vitro and in vivo. Increasing wound SDF-1α via cell-base therapy promoted healing in diabetic mice (−20% increase in healing rates by day 3, p=0.006). SDF-1α increased EC-EPC adhesion and specifically upregulated E-selectin expression in human microvascular EC (2.3-fold increase, p<0.01). This effect was also significant in blood vessels of the experimental mice and resulted in increased wound neovascularization. The regulatory effects of SDF-1α on EC-EPC adhesion and EPC homing were specifically mediated by E-selectin, as the application of E-selectin antagonists significantly inhibited SDF-1α-induced EC-EPC adhesion, EPC homing, wound neovascularization, and wound healing. SDF-1α engineered cell-based therapy promotes diabetic wound healing in mice by specifically upregulating E-selectin expression in mature EC leading to increase EC-EPC adhesion, EPC homing and increased wound neovascularization. These findings provide novel insight into the signals underlying the biological effect of SDF-1α on EPC homing and point to E-selectin as a new target for therapeutic manipulation of EPC trafficking in diabetic wound healing.

### Materials and Methods

HMVECs were isolated from normal human dermis and cultured on plates coated with 1% gelatin. Human EPC were purchased from NDR1, Philadelphia, Pa., and cultured in complete EGM2 medium containing supplements and 5% fetal bovine serum (FBS) (Cambrex Bioscience, Walkersville, Md.). 293, 293T and NIH/3T3 cells were cultured in DMEM (Invitrogen, Carsbad, Calif.) supplemented with 10% FBS. All cells were incubated at 37° C. in 98% humidified air containing 5% CO₂. For cell adhesion and transendothelial migration assays, EPC were labeled with Dil-Ac-LDL (BT-902, Biomedical Technologies, Stoughton, MA) for 4 hours at 37° C., and washed with phosphate-buffered saline (PBS). Subconfluent HMVEC or EPC were stimulated with recombinant human SDF-1α(100 ng/ml) for various times as indicated in the experiments. BSA (100 ng/ml) was used as control.

Wild-type (WT) female C57 BL6 mice at 8-12 weeks of age were purchased from Charles River (Wilmington, Mass.). 10-12-week old NOD (NOD/shilTJ), 8-week old E-sel^{-/-} (B6.129S4-sele^{tmiDmil/J}) mice and 10-12-week old Rosa26 (lacZ⁺) (B6.129S7-GT (ROSA)26 sor/J) mice were purchased from the Jackson Laboratory (Bar Harbor, Me.). For all surgical procedures, mice were anesthetized with an i.p. injection of 80 mg/kg of ketamine and 20 mg/kg xylazine. For bone marrow transplantation experiments, 1×10⁷ bone marrow cells from Rosa26 mice were suspended in 100 μl of PBS and transplanted into E-sel^{-/-} or WT mice (C57 BL6) through tail vein injection.

Streptozocin (STZ, Sigma-Aldrich) diabetes was induced and monitored as previously described (Gallagher et al., J Clin Invest 2007, 117:1249-1259). NOD mice usually developed diabetes within 14-20 weeks. A total of 48 diabetic mice (N=28, STZ-C57; N=20, NOD) were studied. Serum glucose was measured from the mouse tail vein using a glucometer. Once serum glucose reached 250 mg/dl, mice were followed with daily measurements for 3 days prior to use in experiments. Mean serum glucose levels in diabetic mice were 446 mg/dl with a range of 356-512 mg/dl, while mean serum glucose levels in control non-diabetic mice were 122 mg/dl with a range of 96-137 mg/dl. Wounds were induced on the dorsal surface of the mouse back using a 6-mm punch biopsy. Full-thickness skin was removed, exposing the underlying muscle.

Based on prior studies documenting optimal viral transduction efficiency and on projected potential clinical relevance, lentivirus vectors were selected for in vitro studies and adeno-associated virus vectors were selected for in vivo experiments, as tools for manipulating levels of SDF-1α. Human SDF-1α/lenti was constructed by inserting the human or murine SDF-1α genes into pHX vector (Balint et al., J Clin Invest 2005, 115:3166-3176). Control vector, GFP/lenti was constructed as described previously (Liu et al., Cancer Res 2006, 66:4182-4190). Production of pseudotyped lentivirus was achieved by co-transfecting 293 T cells with three plasmids as described (Liu et al., FASEB J 2006, 20:1009-1011). The lentiviruses collected 48 hours post-transfection displayed titers of around 10⁷ transducing units/ml in NIH/3T3 cells. To infect target cells by lentiviruses, cells were exposed for six hours to virus with MOI (multiplicity of infection) 5 in the presence of 4 μg/ml polybrene (Sigma-Aldrich). Cells were then washed, cultured with regular complete medium for two additional days, and analyzed for protein expression by ELISA or pooled for subsequent analysis as indicated in individual experiments. Murine SDF-1α/AAV and control vector, LacZ/AAV were constructed by inserting the murine SDF-1α or LacZ gene into AAV2 vector (Gao et al., Curr Gene Ther 2005, 5:285-297). Production of AAV was achieved by transfecting 293 cells with three plasmids and AAV was purified by heparin chromatography method and titrated. For local wound injections with recombinant AAV, 100 μl of AAV at 10¹² viral unit/ml in PBS was injected into the wound base. For cell-based therapy, bone marrow-derived fibroblasts (BMDFs) were created by culturing murine bone marrow cells on plastic dish in DMEM medium supplemented with 10% FBS for two weeks with medium changes every 3 days. Adherent cells displayed spindle shape and are α-smooth muscle actin⁺ (αSMC⁺) consistent with the myofibroblast phenotype. BMDFs were transduced with lentiviral vectors encoding murine SDF-1α or GFP (as control). Expression of exogenous mSDF-1α in BMDFs was confirmed by ELISA (data not shown). 6-mm punch biopsy skin wounds were created and 1×10⁷ mSDF-1α/BMDFs versus GFP/BMDFs suspended in 100 μl of PBS were injected into the wound.

The Human Adhesion Molecules & ECM RT² Profiler™ PCR Array quantitatively profiles the expression of 84 genes of adhesion molecules and ECM (#PA-011, SABiosciences, Frederick, Md.). Total RNA was extracted from cells using Trizol® (Invitrogen) and cDNA was synthesized using RT² First Strand Kits (SA Biosciences) PCR array was carried out according to the manufacturer's protocol. The threshold cycle (Ct) values were used to plot a standard curve. All samples were normalized to the relative levels of β-actin, and results are expressed as fluorescence intensity in relative levels.

Concentration of SDF-1α in the supernatant of cell culture was measured by Quantikine® SDF-1αELISA kit (DY460, R&D Systems) based on the manufacturer's protocol.

EC-monolayers of HMVEC were cultured in 24-well plate to near confluency and stimulated with recombinant human SDF-1α or BSA at 100 ng/ml for 8 hours. Subsequently, culture medium was replaced with SDF-1α-free EGM2 medium. 1×10⁵ Dil-Ac-LDL-labeled EPC, which were pre-labeled and cultured on 2% agarose-coated plate as suspension for 16 hours, were added into wells and co-cultured with the HMVEC monolayer for 1 hour at 37° C. Unbound EPC were washed out by PBS twice and adherent Dil-Ac-LDL-labeled EPC were measured by fluorescence scanner (GE Typhoon Trio, Piscataway, N.J.) and photographed.

1×10⁴ cells/well of HMVEC were cultured in the upper chamber of a fibronectin (5 μg/ml)-coated 24-transwell insert (8.0-μM pores; Falcon 353097, Becton Dickinson Bedford, Mass.). Before each experiment, monolayer confluency was confirmed by inverted fluorescence microscopy. The HMVEC monolayers were stimulated with recombinant human SDF-1α or BSA at 100 ng/ml for 8 hours. 1×10⁴ Dil-Ac-LDL-labeled EPC, which were pre-labeled as detailed above, were added into the upper chamber in 0.3 mL basal EGM2 medium. 0.6 mL complete EGM2 medium was added to the lower chamber of the transwell. Cells were cultured for 12 hours at 37° C., and EPC traversing from the upper to the lower chamber of the transwell were quantified.

Immunoblotting was performed as described (Liu et al., Mol Cell Biol 2003, 23:14-25). Membranes were probed with antibodies (Abs) to E-selectin (ab-18981) or β-actin (AC-15, Abcam, Cambridge, Mass.). This was followed by probing with HRP-conjugated secondary Ab (Santa Cruz, Santa Cruz, Calif.) and subjected to ECL (Amersham Biosciences, Piscataway, N.J.). Membranes were stripped and re-blotted as required in the individual experiments.

For immunostaining and immunohistochemistry (IHC), 5-1 μm paraffin or frozen sections were processed and were then incubated with FITC-anti-E-selectin (R&D Systems), PE-anti-KDR (Cell Signaling Technology, Danvers, MA) or anti-SDF-1α (sc28876, Santa Cruz) for overnight at 4° C., then incubated with HRP-conjugated secondary antibodies. Immunoreactivity was detected using DAB kit (Dako, Carpinteria, Calif.). The nuclei were counterstained with either DAPI (Vector Labs, Burlingame, Calif.) or hematoxylin (IHC). Negative controls for all antibodies were made by replacing the primary antibodies with non-immunogen, isotype matched Abs from the same manufacturer.

To induce limb ischemia, the full length (about 3-4 mm) of the right femoral artery/vein vascular bundle were ligated and excised (two groups of mice were studied: E-sel^{-/-} mice (n=6) and WT mice (n=6)). The skin was then closed with 5-0 nylon (Ethicon). Limb ischemia was confirmed by Laser Doppler perfusion imaging. Ischemic hindlimb wounds were induced on the ventral surface of the thigh of the mouse using a 4 mm punch biopsy. A full thickness section of skin was removed, exposing the underlying muscle distal to the level of the femoral fold. Recombinant murine SDF-1α protein (R&D Systems) was reconstituted in PBS and injected into the wound base (25 μg/kg) right after the surgery.

Wounds were followed serially with daily digital photographs using an Olympus digital camera. A ruler was included in all photos to allow for calibration of measurements. Images were analyzed using ImageJ software (Imaging Processing and Analysis in Java, National Institutes of Health, MD). Wound area was measured each day, and the wound's percent recovery rate was expressed as [(original wound area minus daily wound area)/(original wound area)] X 100.

Mouse blood vessels were directly labeled in vivo in anesthetized mice by live perfusion using a specially formulated aqueous solution (7 ml/mouse) containing DiI (D-282, Invitrogen/Molecular Probes), which incorporates into endothelial cell membranes upon contact, and was administered via direct intra-cardiac injection prior to animal sacrifice. Seven ml of fixative (4% paraformaldehyde) was injected following Dil perfusion and the entire wound tissue was harvested. The vascular network was visualized by scanning the entire wound tissue to a thickness or depth of 200 μm, using laser scanning confocal microscopy (Vibratome (VT1000S, Leica Microsystems). Vessel density was quantified assessing total number of red Dil-labelled vessels normalized to the entire scanned wound area, using ImageJ software.

Limb perfusion was assessed daily using Laser Doppler Perfusion Imaging (LDI) (Periscan PIM II, Perimed AB, Sweden). The limb was defined as all imaged tissue distal to the femoral fold of the mouse. LDI was performed in a temperature controlled facility with weight based sedation to minimize artifacts due to temperature fluctuations and level of sedation. Relative perfusion data were expressed as the ratio of the ischemic (right) to normal (left) limb blood flow.

1×10⁷ bone marrow cells from 10-12-week old Rosa26 (LacZ⁺) mice were engrafted into E-sel^{-/-} mice (n=6) and WT mice (n=6) through tail vein injection right after creation of ischemic hindlimb wounds (right). The number of LacZ⁺ EPC recruited to wound tissues and integrated into blood vessels in tissue sections were quantified by β-galactosidase assay. Harvested wound tissues were frozen and tissue sections were then incubated with X-gal (Fermentas, Canada) and anti-KDR (ab-2349, Abcam) for 2 hours at room temperature. Sections were counterstained with nuclear fast red (Vector Labs). The number of EPC was quantified by counting β-galactosidase⁺ cells in KDR⁺ vessels in serial sections of wound granulation tissues underlying the excisional wounds at post-operative day 7 (n=3) in 5 random high power fields (HPF, 40×) per section in at least 3 serial sections.

Statistical analysis of differences was performed using ANOVA and 2-tail Student's t-test. Data was analyzed using Microsoft Excel (Microsoft Corp, Redmond, Wash.). Data is expressed as mean±standard error. Values are considered statistically significant when p<0.05.

### Results

SDF-1α-engineered cell-based therapy promotes cutaneous wound neovascularization and diabetic wound healing. The microvasculature in the granulation tissue of cutaneous wounds is physically supported by connective tissue elements elaborated by resident fibroblasts. These fibroblasts provide a unique microenvironment that facilitates and sustains the newly formed vessels. Fibroblasts and their activated counterpart, the myofibroblasts, play a pivotal role in regulating neovascularization in the process of wound healing by synthesis of extracellular matrix (ECM) and secreting various soluble factors (Tomasek et al., Nat Rev Mol Cell Biol 2002, 3:349-363; Kalluri and Zeisberg, Nat Rev Cancer 2006, 6:392-401; Hinz et al., Am J Pathol 2007, 170:1807-1816). It was previously reported that tissue levels of SDF-1α in diabetic murine wounds were significantly decreased, partly due to its down-regulation in myofibroblasts. Myofibroblasts can either arise from the local, resident fibroblasts or from circulating mesenchymal precursors/stem cells (De Wever and Mareel, J Pathol 2003, 200:429-447; Direkze et al., Cancer Res 2004, 64:8492-8495).

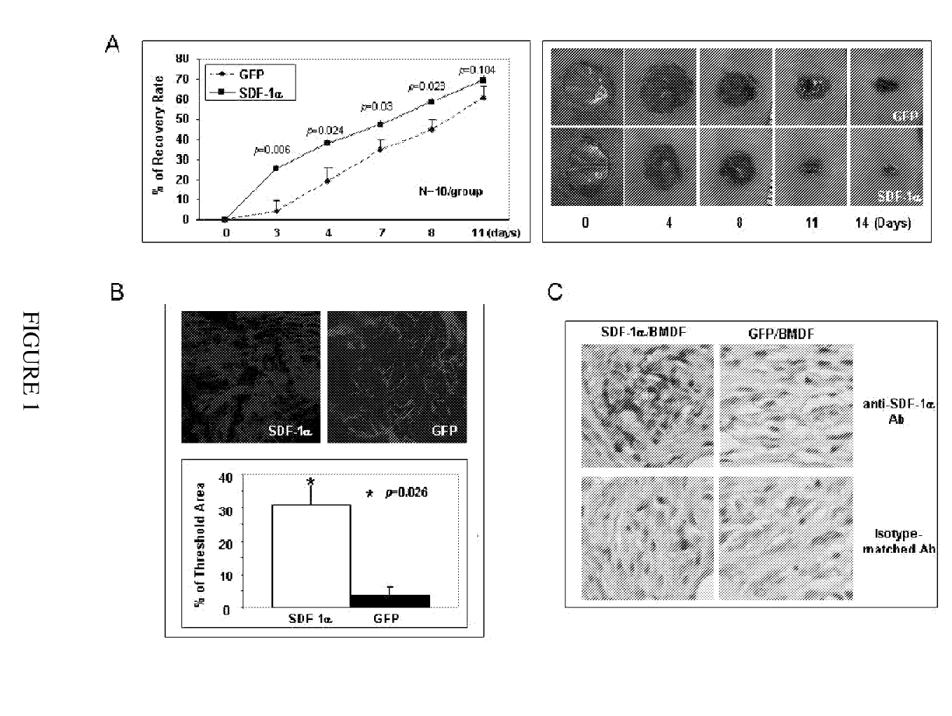

The efficacy of SDF-1α-engineered cell-based therapy for diabetic wound healing was tested. The purpose was to test the pro-healing effect of fibroblast-derived SDF-1α on diabetic wound healing in a genetic (NOD) diabetes murine model. BMDFs were selected, as mature resident cutaneous fibroblasts in patients with diabetes associated wounds are known to be impaired and thus carry little clinical relevance as potential therapeutic vehicle. 1×10⁷ mSDF-1α/BMDFs versus GFP/BMDFs suspended in 100 μl of PBS were injected into the wound. Wounds were photographed daily until closure. Diabetic wounds treated with mSDF-1α/BMDFs were completely healed significantly faster than those injected with GFP/BMDFs (FIG. 1A). Similar pro-healing results were observed in STZ-057 diabetic mice with cutaneous wounds. The naked vector treatment of wounds also showed a pro-healing response but not as pronounced as with the cell-based approach. The most significant difference was observed at day 4 and 5. Correspondingly, significantly more active neovascularization developed in wounds treated with mSDF-1α/BMDFs compared to the control wounds as demonstrated by confocal laser scanning microscopy of wounds after blood vessel perfusion with Dil dye (FIG. 1B). Wounds were harvested and subjected to IHC analysis. Stronger expression of SDF-1α in wounds injected with mSDF-1α/BMDFs versus GFP/BMDFs was confirmed by IHC (FIG. 1C). These results confirmed a pro-healing and pro-angiogenic effects of SDF-1α in diabetic murine models and provided pre-clinical evidence that SDF-1α-engineered cell-based therapy may serve as a novel tool for the treatment of diabetic wounds.

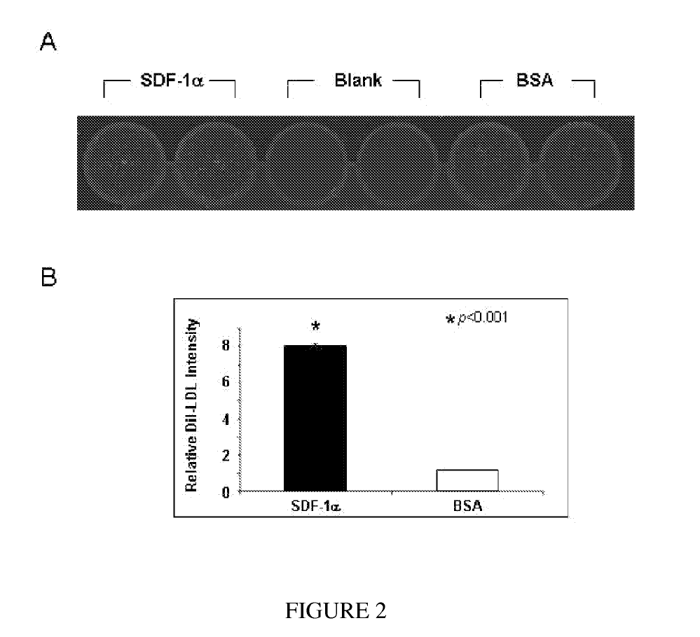

Enhanced adhesion of human EPC to SDF-1α-stimulated EC monolayer in vitro. To test the hypothesis that the effect of SDF-1α on EPC homing is mediated by regulating adhesion molecule(s) on mature endothelial cells, whether or not SDF-1α stimulation could make an EC monolayer more likely to support adhesion of EPC in an in vitro cell-cell adhesion assay was examined. Subconfluent HMVEC cultured in 24-well plates were stimulated with recombinant human SDF-1α or BSA. Dil-Ac-LDL-labeled EPC were added into wells and co-cultured with EC-monolayer for 1 hour. Unbound EPC were washed out by PBS twice and adherent Dil-Ac-LDL-labeled EPC were measured by fluorescence scanner (FIG. 2A). There was approximately an 8-fold increase in the number of EPC adherent to the SDF-1α-stimulated EC monolayers compared to BSA-treated control (FIG. 2B). This data indicated that SDF-1α stimulation promotes direct EPC-EC adhesion, suggesting that the expression of certain adhesion molecule(s) on EC may be specifically regulated by SDF-1α.

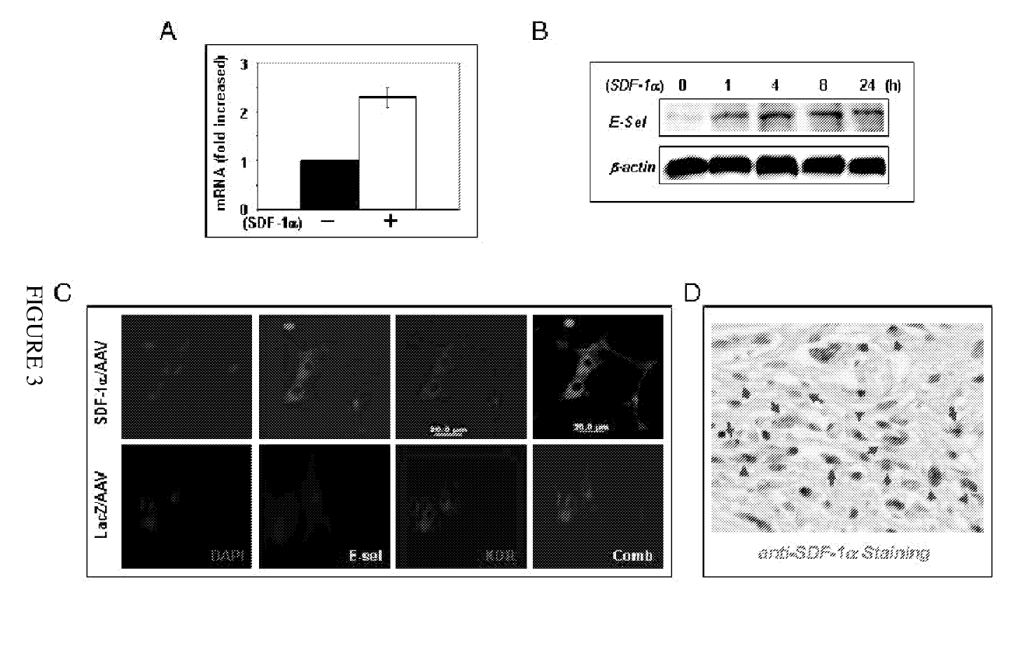

SDF-1α stimulation up-regulates expression of E-selectin in EC monolayers (in vitro) and wound capillary endothelium of mice (in vivo). To identify the adhesion molecule(s) up-regulated by SDF-1α in EC monolayers, a RT² Profiler™ PCR array was carried out. Subconfluent HMVEC were stimulated with recombinant human SDF-1α protein versus BSA at 100 ng/ml for 4 hours. Cells were harvested and total RNA was extracted and subjected for RT² Profiler™ PCR array. Of the 84 genes tested in the array, 13 were up-regulated (>1.5-fold) while 20 down-regulated (<1.5-fold) and 51 were unaltered (Table 1). Notably, the expression of E-selectin gene was increased about 2.3-fold upon SDF-1α stimulation (FIG. 3A). To validate the observed up-regulation of mRNA of E-selectin, immunoblotting analyses were conducted and an up-regulated protein expression of E-selectin in SDF-1α-stimulated compared to BSA-treated EC monolayers (FIG. 3B) was confirmed. To further validate the up-regulation of E-selectin by SDF-1α in mature endothelial cells in vivo, vessels in diabetic NOD mice wounds injected with mSDF-1α/AAV versus lacZ/AAV were examined by immunostaining. E-selectin was stained with FITC-conjugated anti-E-selectin Ab and EC with PE-conjugated anti-KDR Ab. EC of blood vessels in diabetic murine wounds injected with mSDF-1α/AAV expressed stronger E-selectin (yellow) compared to those injected with LacZ/AAV (FIG. 3C). Increased tissue levels in mSDF-1α/AAV was demonstrated by IHC (FIG. 3D). These experiments identified E-selectin as one key adhesion molecule that is up-regulated as a down-stream target of SDF-1α in vascular EC.

TABLE 1
Genes up- and down-regulated in 4 hours upon
SDF- 1α stimulation in HMVECs.

| | Fold of |
|---|---|
| Up-Regulated | mRNA changes |
| CO11A1/COLL6 | 2.14 |
| CD49D/IA4 | 1.87 |
| CD49f/ITGA6B | 2.14 |
| CD51/DKFZp686A08142 | 2.3 |
| LAMB2 | 2 |
| CLG1/HNC | 3.73 |
| CD62E/ELAM | 2.3 |
| CD62/CD62P | 2.3 |
| DYT11/ESG | 1.74 |
| BNSP/BSPI | 2.14 |
| THBS/TSP | 1.74 |
| CLGI/EPA | 1.74 |
| CD106/DKFZp779G2333 | 2.14 |
| Down-Regulated | |
| CSPG2/DKFZp686K06110 | −2.14 |
| CCN2/HCS24 | −3.03 |
| CTNNB/DKFZp686D02253 | −2.00 |
| CAS/CTNND | −3.73 |
| CIG/DKFZp686F10164 | −3.48 |
| BB2/CD54 | −2.82 |
| CD49a/VLA1 | −4.90 |
| BR/CD49B | −3.03 |
| CD49e/FNRA | −5.30 |
| CD29/FNRB | −2.30 |
| CD61/GP3A | −2.30 |
| FLJ26658 | −8.57 |
| LAMM | −2.82 |
| CLM | −2.82 |
| LAMNB1 | −5.28 |
| MMP-X2/MT-MMP2 | −6.06 |
| CLG4/CLG4A | −4.28 |
| ON | −2.63 |
| CAR/CMAR | −4.00 |
| TSP3 | −2.46 |

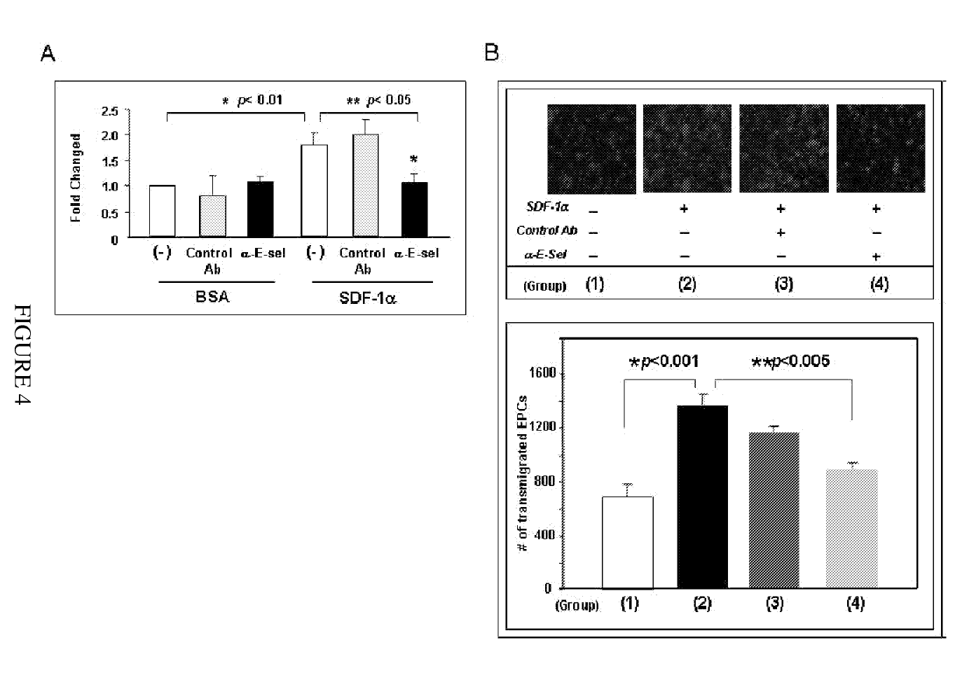

Up-regulated E-selectin is responsible for mediating SDF-1α-enhanced EC-EPC interaction and EPC transendothelial migration. To study whether up-regulated E-selectin is responsible for mediating SDF-1α-enhanced EC-EPC adhesion, the effect of E-selectin antagonist on EPC adhesion to SDF-1α-stimulated EC monolayer in vitro was tested. Subconfluent HMVEC cultured in 24-well plate were stimulated with recombinant human SDF-1α or BSA at 100 ng/ml for 8 hours and culture medium was replaced with SDF-1α-free EGM2 medium containing either E-selectin neutralizing Ab or isotype-matched control Ab (2 μg/ml) and incubated for 15 minutes at 37° C. before adding EPC. Subsequently, 1×10⁵ Dil-Ac-LDL-labeled EPC, which were pre-labeled as detailed above, were added into the wells and co-cultured with the EC-monolayer for 1 hour at 37° C. Unbound EPC were washed out by PBS twice and adherent Dil-Ac-LDL-labeled EPC were measured by fluorescence scanner. The addition of E-selectin neutralizing Ab significantly inhibited the number of EPC adherent to SDF-1α-stimulated EC monolayers whereas control Ab had no significant effect (FIG. 4A). To further investigate the biological function of E-selectin in mediating a specific interaction between EPC and the EC monolayer, whether or not increased adhesion between EPC and the EC monolayer results in more EPC transmigrating through the EC monolayers was examined, and if yes, whether or not this transmigration effect is E-selectin-dependent was examined. EPC transendothelial migration was tested in an in vitro transwell system. HMVEC monolayers were cultured in the upper chamber of transwell inserts in the presence of γhSDF-1α or BSA. Fifteen minutes prior to adding EPC into the insert, EGM2 medium in the lower chamber was replaced with fresh medium containing γhSDF-1α or BSA, respectively. 1×10⁵ Dil-Ac-LDL-labeled EPC suspension prepared as described above were added into the insert and cultured for overnight at 37° C. Compared to BSA-treated EC monolayers, SDF-1α-stimulated EC monolayers showed significantly increased EPC transmigration from the upper to the lower transwell chamber (FIG. 4B). Importantly, this effect is, at least partially, E-selectin-dependent, since blockade of E-selectin by adding neutralizing Ab (2 μg/ml) to the transwell was able to significantly inhibit EPC transendothelial migration (FIG. 4B). Overall, these results demonstrated that up-regulated E-selectin is responsible for mediating SDF-1α-enhanced EC-EPC adhesion and EPC transendothelial migration.

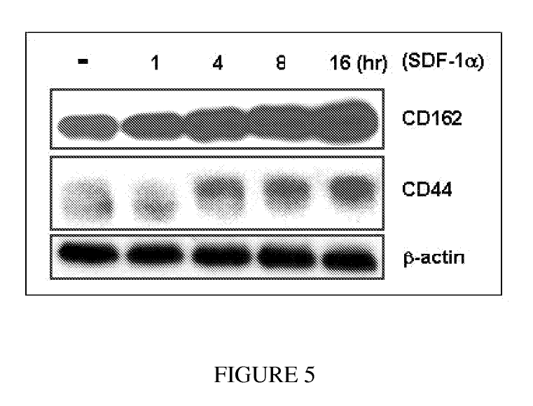

SDF-1α stimulation up-regulates expression of E-selectin ligands in EPC. Ultimately, the direct interaction between EC lining the wound's capillaries and the circulating EPC would depend upon mutually relevant counterparts of adhesion molecules on the surface of both cell types. It was hypothesized that SDF-1α might induce expression of relevant adhesion molecule(s) on both EC and EPC. Based on the results detailed above that SDF-1α-induced E-selectin on EC mediates direct EC-EPC interactions, the expression of two E-selectin ligands, CD44 and CD162 (PSGP-1), on EPC in response to SDF-1α stimulation was examined. Subconfluent human EPC were stimulated with γhSDF-1α or BSA, respectively, for various periods of time. Cells were harvested and subjected to immunoblotting analyses. Unstimulated EPC expressed strong basal levels of CD162 and low levels of CD44. SDF-1α stimulation up-regulated expression of both CD162 and CD44 in EPC (FIG. 5). Induction of CD162 and CD44 was first observed in 4 hours upon SDF-1α stimulation and continued to increase until 16 hours. These experiments confirmed that EPC express basal levels of E-selectin ligands and show that SDF-1α is able to up-regulate the expression of these E-selectin ligands in EPC, consistent with the overall hypothesis that the soluble factor, SDF-1α, mediates EPC homing effects by specifically regulating the profile of adhesion molecules on these two key cell types, the mature endothelial cell and the EPC.

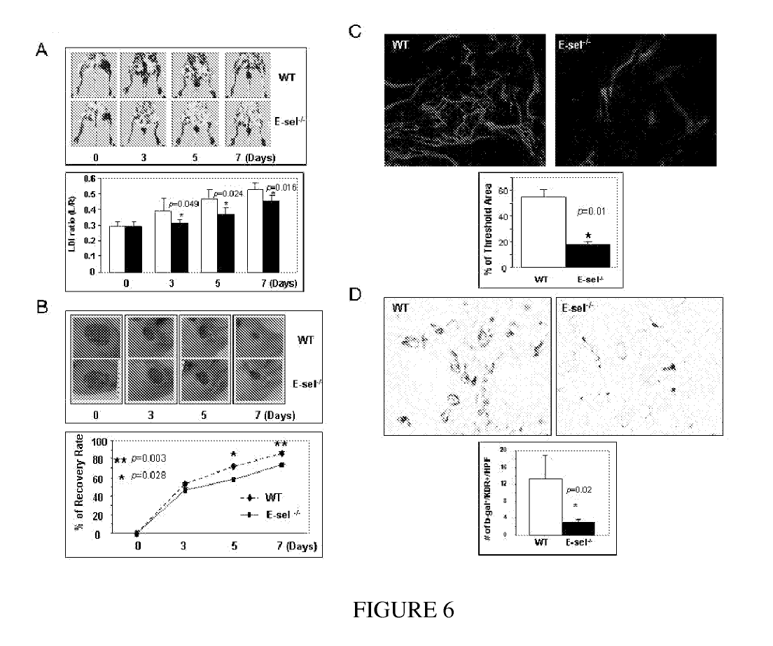

E-selectin is required for mediating effects of SDF-1α on EPC homing, neovascularization and wound healing in murine ischemic hindlimb and cutaneous wound model. To study the specific biological importance of SDF-1α-induced E-selectin in EPC homing, neovascularization, and cutaneous wound healing, a loss-of-function approach in combination with bone marrow transplantation was employed. A murine model of unilateral hindlimb ischemia via femoral ligation/excision and subsequent bilateral 4-mm cutaneous excisional wounding was created in E-sel^{-/-} versus WT mice. Recombinant murine SDF-1α was administrated to wounds via direct wound injections. Further treatment was provided by 1×10⁷ bone marrow cells from Rosa26(LacZ⁺) mice injected into mice via the tail vein, to quantify (partially) the contribution of EPC homing to neovascularization and wound healing. LDI was used to confirm post-operative limb ischemia, and monitor and quantify the spontaneous restoration of hindlimb blood flow via mean perfusion measured over time. Daily wound area measurements were obtained via digital photography. Neovascularization in wound tissues was evaluated by blood vessel Dil perfusion and subsequent laser scanning confocal microscopy in harvested wound tissues in half of experimental mice at day 7. In the remaining mice, the wounds were harvested and subjected to IHC. EPC recruited to wound tissues and incorporated into blood vessels were detected by double staining with 13-gal (blue) and anti-KDR. Compared to WT mice, ischemic hindlimbs in E-sel^{-/-} mice showed delayed improvements in perfusion over time as indicated by the significantly lower mean flux measurements (FIG. 6A). Consistently, ischemic wounds in E-sel^{-/-} mice had significantly slower closure rate that in WT mice (FIG. 6B). In addition, ischemic wounds in E-sel^{-/-} mice were much more poorly vascularized compared to those in WT mice (FIG. 6C). The poor neovascularization in ischemic wounds of E-sel^{-/-} mice was, at least partially, related to fewer EPC homing, since significantly less LacZ⁺ cells were detected in blood vessels of ischemic wounds in the E-sel^{-/-} mice compared to the WT mice (FIG. 6D). These in vivo experiments demonstrated that E-selectin is required for mediating SDF-1α-induced EPC homing, neovascularization and wound healing.

The experiments described herein demonstrated that SDF-1α specifically upregulates the expression of E-selectin in mature EC and E-selectin ligands on EPC, thereby mediating EC-EPC adhesion and EPC homing. These findings provide profound and novel insight into the molecular mechanism underlying the biological effect of SDF-1α on EPC homing and point to E-selectin as a new target for therapeutic manipulation of EPC homing in diabetes-associated microangiopathy and delayed wound healing (as well as other ischemic wounds). The modulation of E-selectin and its ligands in EC and EPC offers an opportunity to not only regulate the EPC-associated direct angiogenic response, but also, the associated direct and indirect effects on wound healing that are intrinsically relevant to the unsolved clinical problem of delayed diabetes-associated cutaneous wound healing.

### Other Embodiments

Any improvement may be made in part or all of the compositions, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. For example, although the experiments described herein involve diabetic wounds, the compositions, kits and methods described herein can find use in a number of other therapeutic and prophylactic applications, including any ischemic wound. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of promoting healing of a diabetic wound in a diabetic subject, the method comprising the steps of:
providing a therapeutically effective amount of a composition comprising a pharmaceutically acceptable carrier and at least one therapeutic agent selected from the group consisting of: E-selectin protein, a nucleic acid encoding E-selectin protein, and an agent that specifically upregulates E-selectin expression; and
administering the composition to the subject under conditions such that migration of bone marrow-derived progenitor cells to the wound is increased in the subject.

2. The method of claim 1, wherein the composition is administered orally, topically, or intravenously.

3. The method of claim 1, wherein the composition is administered directly to the wound or a site adjacent to the wound.

4. The method of claim 1, wherein the bone marrow-derived progenitor cells comprise endothelial progenitor cells (EPC).

5. The method of claim 1, wherein administration of the composition to the subject results in accelerated wound healing.

6. The method of claim 1, wherein the composition comprises E-selectin protein or a nucleic acid encoding E-selectin protein, and an agent that specifically upregulates E-selectin expression, wherein the agent that specifically upregulates E-selectin expression is SDF-1α protein or a nucleic acid encoding SDF-1α protein.

7. The method of claim 1, further comprising the step of administering to the subject hyperbaric oxygen treatment.

8. A method of upregulating E-selectin expression in a diabetic subject having a diabetic wound, the method comprising administering to the diabetic subject a composition comprising at least one rAAV virion comprising a polynucleotide encoding E-selectin, the polynucleotide interposed between a first AAV inverted terminal repeat and second AAV inverted terminal repeat, the composition in an amount effective to upregulate E-selectin expression, induce migration of bone marrow-derived progenitor cells to the wound, and accelerate healing of the wound in the subject

9. The method of claim 8, wherein the at least one rAAV virion comprises serotype 2 capsid proteins.

10. The method of claim 8, wherein the composition is administered directly to the wound or a site adjacent to the wound.

11. The method of claim 8, wherein the bone marrow-derived progenitor cells comprise EPC.

12. The method of claim 8, further comprising the step of administering SDF-1α protein or a nucleic acid encoding SDF-1α protein to the subject.

13. The method of claim 8, further comprising the step of administering to the subject hyperbaric oxygen treatment.

14. A method of promoting healing of a diabetic wound in a diabetic subject, the method comprising the steps of:
providing a composition comprising a pharmaceutically acceptable carrier and a plurality of bone marrow-derived progenitor cells, wherein the bone marrow-derived progenitor cells comprise a polynucleotide encoding E-selectin; and
administering the composition to the subject in an amount effective to increase migration of bone marrow-derived progenitor cells to the wound and accelerate healing of the wound in the subject.

15. The method of claim 14, wherein the polynucleotide encoding E-selectin is comprised within a viral vector.

16. The method of claim 15, wherein the viral vector is comprised within a viral particle.

17. The method of claim 16, wherein the viral vector is an rAAV vector and the viral particle is an AAV particle.

18. The method of claim 14, wherein the composition further comprises SDF-1α protein or a nucleic acid encoding SDF-1α protein.

19. The method of claim 14, wherein the composition is administered directly to the wound or a site adjacent to the wound.

20. The method of claim 14, wherein the bone marrow-derived progenitor cells comprise EPC.

21. The method of claim 14, further comprising the step of administering to the subject hyperbaric oxygen treatment.

22. A kit for treating at least one diabetic wound in a mammalian subject, the kit comprising:
(a) a therapeutically effective amount of a composition comprising a pharmaceutically acceptable carrier and at least one therapeutic agent selected from the group consisting of: E-selectin protein, a nucleic acid encoding E-selectin protein, and an agent that specifically upregulates E-selectin expression; and
(b) instructions for use.

23. The kit of claim 1, wherein the composition comprises E-selectin protein or a nucleic acid encoding E-selectin protein, and an agent that specifically upregulates E-selectin expression, wherein the agent that specifically upregulates E-selectin expression is SDF-1α protein or a nucleic acid encoding SDF-1α protein.

24. The kit of claim 1, wherein the instructions for use comprise instructions for administering hyperbaric oxygen treatment to the subject.
